(19) United States
(12) Patent Application Publication
(10) Pub. No.: US 2024/0239760 A1
(43) Pub. Date: Jul. 18, 2024

(54) PPARG INVERSE AGONISTS AND USES THEREOF

(71) Applicant: Flare Therapeutics Inc., Cambridge, MA (US)

(72) Inventors: James E. Audia, Chicago, IL (US); Jonathan E. Wilson, Arlington, MA (US)

(21) Appl. No.: 18/387,913

(22) Filed: Nov. 8, 2023

Related U.S. Application Data
(60) Provisional application No. 63/423,571, filed on Nov. 8, 2022.

(51) Int. Cl. C07D 263/57; A61K 31/4184; A61K 31/4192; A61K 31/423; A61K 31/428; A61K 31/437; A61K 31/4439; A61K 31/444; A61K 31/472; C07D 217/04; C07D 235/18; C07D 249/20; C07D 277/66; C07D 401/12; C07D 413/04; C07D 413/12; C07D 413/14; C07D 417/12; C07D 471/04

(52) U.S. Cl. CPC C07D 263/57; A61K 31/4184; A61K 31/4192; A61K 31/423; A61K 31/428; A61K 31/437; A61K 31/4439; A61K 31/444; A61K 31/472; C07D 217/04; C07D 235/18; C07D 249/20; C07D 277/66; C07D 401/12; C07D 413/04; C07D 413/12; C07D 413/14; C07D 417/12; C07D 471/04

(57) ABSTRACT

Provided are compounds of Formula (I):

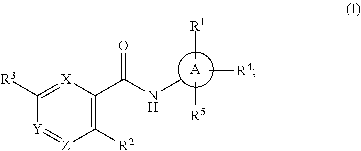

and pharmaceutically acceptable salts and compositions thereof, which are useful for treating a variety of conditions associated with PPARG.

### RELATED APPLICATIONS

This application claims the benefit of priority to U.S. Provisional Application No. 63/423,571, filed Nov. 8, 2022, the entire contents of which is incorporated herein by reference.

### REFERENCE TO ELECTRONIC SEQUENCE LISTING

The application contains a Sequence Listing which has been submitted electronically in .XML format and is hereby incorporated by reference in its entirety. Said .XML copy, created on Mar. 6, 2024, is named “132266-00702.xml” and is 5,096 bytes in size. The sequence listing contained in this .XML file is part of the specification and is hereby incorporated by reference herein in its entirety.

### BACKGROUND

PPARgamma (PPARG) is a type II ligand-dependent nuclear hormone receptor (belonging to the PPAR nuclear receptor subfamily) that functions as an obligate heterodimer with retinoid X receptors (RXRs). PPARG is predominantly expressed in adipose tissue, colon, macrophages and the luminal layers of the urothelium. PPARG is known as a master regulator of adipogenesis, functioning to regulate adipocyte differentiation, fatty acid storage and glucose metabolism. PPARG has also been shown to play an important role in the metabolism and inflammation of macrophages, where it is induced by IL4 and controls glutamine metabolism. In the normal urothelium, PPARG is critical for its homeostasis and regeneration.

The role for PPARG in cancer was originally inferred from genomic studies that identified a PAX8-PPARG chromosomal rearrangement in follicular thyroid carcinomas. More recently, PPARG has been found to be over-expressed and genetically altered in the luminal subtype of urothelial cancer. This consistent with reports that long-term use of PPARG agonists is associated with an increased incidence of urothelial cancer. Most urothelial cancers are urothelial carcinoma, which are classified as either non-muscle-invasive urothelial cancer (NMIUC, 70%), muscle-invasive urothelial cancer (MIUC, 25%) or metastatic urothelial cancer (MUC, 5%). MIUC is usually diagnosed de novo but may arise from the 10 to 20% of NMIUC cases that eventually progress. MIUC is a heterogeneous and aggressive disease, associated with a five-year survival rate of 60% for patients with localized disease and less than 10% for patients with distant metastases. Molecular understanding of NMIUC and MIUC has improved significantly, including the association between molecular subtypes and urothelial differentiation. Several molecular classes of MIUC have been proposed, whereby an activated PPARG signature features prominently in the luminal subtypes. First-line treatment is chemotherapy with several options in chemo-ineligible or second line, but treatment options are limited with poor overall survival rates.

The need exists to develop effective PPARG modulators for treating cancers such as NMIUC, MIUC, and MUC, and related conditions.

### SUMMARY

Provided herein are compounds having the Formula I:

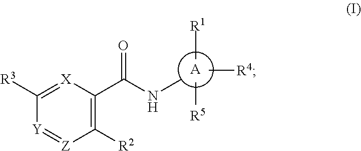

and pharmaceutically acceptable salts and compositions thereof, wherein R¹, R², R³, R⁴, R⁵, X, Y, and Z are as described herein. In one aspect, the disclosed compounds of Formula I and pharmaceutically acceptable salts thereof modulate PPARG (e.g., as agonists such as inverse agonists, and are useful in a variety of therapeutic applications such as, for example, in treating cancer. As such, their uses for treating diseases responsive to the inhibition of PPARG are included.

Pharmaceutical compositions comprising the compounds and pharmaceutically acceptable salts of the disclosed compounds of Formula I, as well as methods for their preparation are also included.

### DETAILED DESCRIPTION

### 1. General Description of Compounds

In a first embodiment, provided herein is a compound of Formula I:

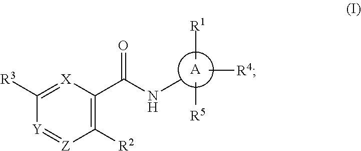

or a pharmaceutically acceptable salt thereof, wherein
X, Y, and Z are each independently N or —CR⁶;
ring A is a fused bicyclic heteroaryl;
R¹, R⁴, and R⁵ are each independently selected from hydrogen, halo, (C₁-C₄)alkyl, (C₁-C₄)alkoxy, halo(C₁-C₄)alkyl, halo(C₁-C₄)alkoxy, —(C₁-C₄)alkylOR^{a}, —(C₁-C₄)alkylC(O)R^{a}, —(C₁-C₄)alkylC(O)OR^{a}, —C(O)NR^{a}R^{b}, —(C₁-C₄)alkylC(O)NR^{a}R^{b}, —C(O)R^{a}, —C(O)OR^{a}, —NR^{a}R^{b}, —(C₁-C₄)alkylNR^{a}R^{b}, —C(O)NR^{a}SO₃H, —NR^{a}C(O)R^{b}, —NR^{a}C(O)OR^{b}, —NR^{a}C(S)OR^{b}, —N^{c}C(O)N^{a}R^{b}, —N^{c}C(S)NR^{a}R^{b}, —NR^{c}S(O)₂NR^{a}R^{b}, —C(S)R^{a}, —S(O)₂R^{a}, —S(O)R^{a}, —C(S)OR^{a}, —C(S)NR^{a}R^{b}, —NR^{a}C(S)R^{b}, —SR^{a}, phenyl, 4- to 6-membered heterocyclyl, and 5- to 7-membered heteroaryl, wherein each of said phenyl, 4- to 6-membered heterocyclyl, and 5- to 7-membered heteroaryl are optionally and independently substituted with 1 to 3 groups selected from R⁷;
R² is halo, —SR^{g}, —SOR^{g}, —SO₂R^{g}, —SO(═NR^{g})R^{h}, —OR^{g}, or —X¹Rⁱ;
R³ is cyano or nitro;
R⁶ is hydrogen, halo, (C₁-C₄)alkyl, (C₁-C₄)alkoxy, or hydroxyl;
R⁷ is selected from halo, (C₁-C₄)alkyl, halo(C₁-C₄)alkyl, (C₁-C₄)alkoxy, halo(C₁-C₄)alkoxy, nitro, oxo, cyano, —(C₁-C₄)alkylOR^{d}, —(C₁-C₄)alkylC(O)R^{d}, —(C₁-C₄)alkylC(O)OR^{d}, —C(O)NR^{d}R^{e}, —(C₁-C₄)alkylC(O)NR^{d}R^{e}, —C(O)R^{d}, —C(O)OR^{d}, —NR^{d}R^{e}, —(C₁-C₄)alkylNR^{d}R^{e}, —C(O)NR^{d}SO₃H, —N^{c}C(O)R^{e}, —N^{c}C(O)OR^{e}, —N^{c}C(S)OR^{e}, —NR^{f}C(O)N^{d}R^{e}, —NR^{f}C(S)NR^{d}R^{e}, —NR^{f}S(O)₂N^{d}R^{e}, —C(S)R^{d}, —S(O)₂R^{d}, —S(O)R^{d}, —C(S)OR^{d}, —C(S)NR^{d}R^{e}, —N^{c}C(S)R^{e}, and —SR^{d};

X¹ is S, SO, SO₂, or —SONH;

R^{a}, R^{b}, R^{e}, R^{d}, R^{e}, R^{f}, R^{g}, and R^{h} are each independently hydrogen, halo(C₁-C₄)alkyl, or a (C₁-C₄)alkyl optionally substituted with 1 or 2 —NR′R″ groups wherein R′ and R″ are each independently selected from hydrogen, (C₁-C₄)alkyl, and halo(C₁-C₄)alkyl; and
Rⁱ is (C₁-C₄)alkyl, (C₁-C₄)alkoxy, or halo(C₁-C₄)alkyl.

### 2. Definitions

When used in connection to describe a chemical group that may have multiple points of attachment, a hyphen (—) designates the point of attachment of that group to the variable to which it is defined. For example, —NR^{b}C(O)OR″ and —NR^{b}C(S)OR mean that the point of attachment for this group occurs on the nitrogen atom.

The terms “halo” and “halogen” refer to an atom selected from fluorine (fluoro, —F), chlorine (chloro, —Cl), bromine (bromo, —Br), and iodine (iodo, —I).

The term “alkyl” when used alone or as part of a larger moiety, such as “haloalkyl”, and the like, means saturated straight-chain or branched monovalent hydrocarbon radical.

“Alkoxy” means an alkyl radical attached through an oxygen linking atom, represented by —O-alkyl. For example, “(C₁-C₄)alkoxy” includes methoxy, ethoxy, proproxy, and butoxy.

The term “haloalkyl” includes mono, poly, and perhaloalkyl groups where the halogens are independently selected from fluorine, chlorine, bromine, and iodine.

“Haloalkoxy” is a haloalkyl group which is attached to another moiety via an oxygen atom such as, e.g., —OCHF₂ or —OCF₃.

The term oxo means the group ═O.

The term “heteroaryl” used alone or as part of a larger moiety refers to a 5- to 12-membered aromatic radical containing 1-4 heteroatoms selected from N, O, and S. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, triazinyl, tetrazinyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, imidazopyridinyl, benzooxazolyl, benzooxodiazolyl, indazolyl, benzimidazolyl, benzthiazolyl, quinolyl, quinazolinyl, quinoxalinyl, pyrrolopyridinyl, pyrrolopyrimidinyl, pyrazolopyridinyl, thienopyridinyl, thienopyrimidinyl, indolizinyl, purinyl, naphthyridinyl, and pteridinyl. It will be understood that when specified, optional substituents on a heteroaryl group may be present on any substitutable position and, include, e.g., the position at which the heteroaryl is attached.

The term “heterocyclyl” means a 5- to 12-membered saturated or partially unsaturated heterocyclic ring containing 1 to 4 heteroatoms independently selected from N, O, and S. It can be monocyclic, bicyclic (e.g., a bridged, fused, or spiro bicyclic ring), or tricyclic. A heterocyclyl ring can be attached to its pendant group at any heteroatom or carbon atom that results in a stable structure. Examples of such saturated or partially unsaturated heterocyclic radicals include, without limitation, tetrahydrofuranyl, tetrahydrothienyl, terahydropyranyl, pyrrolidinyl, pyridinonyl, pyrrolidonyl, piperidinyl, oxazolidinyl, pipcrazinyl, dioxanyl, dioxolanyl, morpholinyl, dihydrofuranyl, dihydropyranyl, dihydropyridinyl, tetrahydropyridinyl, dihydropyrimidinyl, oxetanyl, azetidinyl and tetrahydropyrimidinyl. A heterocyclyl group may be mono- or bicyclic. The term “heterocyclyl” also includes, e.g., unsaturated heterocyclic radicals fused to another unsaturated heterocyclic radical or aryl or heteroaryl ring, such as for example, tetrahydronaphthyridine, indolinone, dihydropyrrolotriazole, imidazopyrimidine, quinolinone, dioxaspirodecane. It will also be understood that when specified, optional substituents on a heterocyclyl group may be present on any substitutable position and, include, e.g., the position at which the heterocyclyl is attached (e.g., in the case of an optionally substituted heterocyclyl or heterocyclyl which is optionally substituted).

The term “spiro” refers to two rings that shares one ring atom (e.g., carbon).

The term “fused” refers to two rings that share two adjacent ring atoms with one another.

The term “bridged” refers to two rings that share three ring atoms with one another.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “effective amount” or “therapeutically effective amount” refers to an amount of a compound described herein that will elicit a desired or beneficial biological or medical response of a subject e.g., a dosage of between 0.01-100 mg/kg body weight/day.

### 3. Compounds

In a second embodiment, the compound of Formula I is of the Formula II:

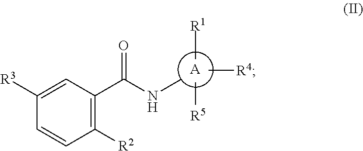

or a pharmaceutically acceptable salt thereof, wherein the remaining variables are as described above for Formula I. Alternatively, as part of a second embodiment, the compound of Formula I is of the Formula III:

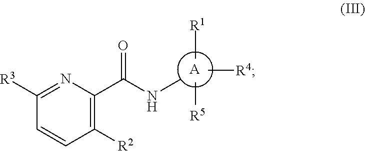

or a pharmaceutically acceptable salt thereof, wherein the remaining variables are as described above for Formula I. In another alternative, as part of a second embodiment, the compound of Formula I is of the Formula IV:

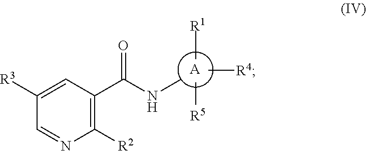

or a pharmaceutically acceptable salt thereof, wherein the remaining variables are as described above for Formula I. In another alternative, as part of a second embodiment, the compound of Formula I is of the Formula V:

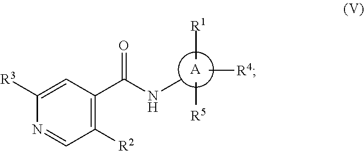

or a pharmaceutically acceptable salt thereof, wherein the remaining variables are as described above for Formula I.

In a third embodiment, R² in the compound of any one of Formula I to V is halo, —S(C₁-C₄)alkyl, —SO(C₁-C₄)alkyl, —SO₂(C₁-C₄)alkyl, or —O(C₁-C₄)alkylN[(C₁-C₄)alkyl]₂, wherein the remaining variables are as described above for Formula I. Alternatively, as part of a third embodiment, R² in the compound of any one of Formula I to V is chloro, —SCH₃, —SOCH₃, —SO₂CH₃, or —O(CH₂)₂N(CH₃)₂, wherein the remaining variables are as described above for Formula I. In another alternative, as part of a third embodiment, R² in the compound of any one of Formula I to V is chloro, wherein the remaining variables are as described above for Formula I.

In a fourth embodiment, R³ in the compound of any one of Formula I to Vis cyano, wherein the remaining variables are as described above for Formula I or the third embodiment.

In a fifth embodiment, ring A in the compound of any one of Formula I to V is selected from

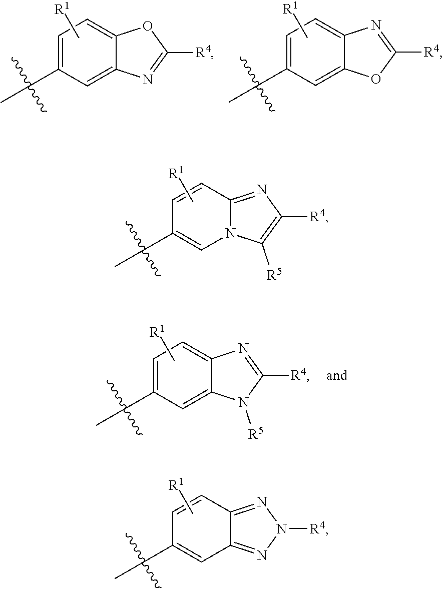

wherein the remaining variables are as described above for Formula I or the third or fourth embodiments. Alternatively, as part of a fifth embodiment, ring A in the compound of any one of Formula I to V is

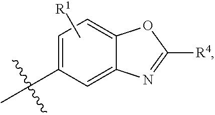

wherein the remaining variables are as described above for Formula I or the third or fourth embodiments.

In a sixth embodiment, R¹ in the compound of any one of Formula I to V is hydrogen or halo, wherein the remaining variables are as described above for Formula I or any one of the third to fifth embodiments. Alternatively, as part of a sixth embodiment, R¹ in the compound of any one of Formula I to V is hydrogen, fluoro, or chloro, wherein the remaining variables are as described above for Formula I or any one of the third to fifth embodiments. In another alternative, as part of a sixth embodiment, R¹ in the compound of any one of Formula I to V is hydrogen, wherein the remaining variables are as described above for Formula I or any one of the third to fifth embodiments.

In a seventh embodiment, R⁵ in the compound of any one of Formula I to V is hydrogen or (C₁-C₄)alkyl, wherein the remaining variables are as described above for Formula I or any one of the third to sixth embodiments. Alternatively, as part of a seventh embodiment, R⁵ in the compound of any one of Formula I to V is CH₃, wherein the remaining variables are as described above for Formula I or any one of the third to sixth embodiments.

In an eighth embodiment, R⁴ in the compound of any one of Formula I to V is phenyl or 5- to 7-membered heteroaryl, each optionally substituted with 1 to 3 groups selected from R⁷, wherein the remaining variables are as described above for Formula I or any one of the third to seventh embodiments. Alternatively, as part of an eighth embodiment, R⁴ in the compound of any one of Formula I to V is phenyl or pyridinyl, each optionally substituted with 1 to 3 groups selected from R⁷, wherein the remaining variables are as described above for Formula I or any one of the third to seventh embodiments. In another alternative, as part of an eighth embodiment, R⁴ in the compound of any one of Formula I to V is phenyl optionally substituted with 1 to 3 groups selected from R⁷, wherein the remaining variables are as described above for Formula I or any one of the third to seventh embodiments.

In a ninth embodiment, R⁷ in the compound of any one of Formula I to V is selected from halo, (C₁-C₄)alkyl, and (C₁-C₄)alkoxy, wherein the remaining variables are as described above for Formula I or any one of the third to eighth embodiments. Alternatively, as part of a ninth embodiment, R⁷ in the compound of any one of Formula I to V is selected from bromo, fluoro, CH₃, CH₂CH₃, and OCH₃, wherein the remaining variables are as described above for Formula I or any one of the third to eighth embodiments.

Compounds having the Formula I are further disclosed in the Exemplification and are included in the present disclosure. Pharmaceutically acceptable salts thereof as well as the neutral forms are included.

### 4. Uses, Formulation and Administration

The compounds and compositions described herein are generally useful for modulating the activity of PPARG. In some aspects, the compounds, pharmaceutical acceptable salts, and pharmaceutical compositions described herein inhibit the activity PPARG. In some aspects, the compounds and pharmaceutical acceptable salts disclosed herein are agonists of PPARG. In some aspects, the compounds and pharmaceutical acceptable salts disclosed herein are agonists of PPARG. In some aspects, the compounds and pharmaceutical acceptable salts disclosed herein are inverse agonists of PPARG. In one aspect, “inverse-agonists” refer to agents that bind to the same receptor binding site as a agonist (e.g., the binding site of a nuclear receptor such as PPARG) and not only antagonizes the effects of an agonist but, moreover, exerts the opposite effect by suppressing spontaneous receptor signaling (when present).

In some aspects, the compounds and pharmaceutical acceptable salts disclosed herein overcome the activated state of PPARG function resulting from alteration in PPARG activity (mutation, amplification or overexpression) or from RXRA activating mutations. In some aspect, the compounds and pharmaceutical acceptable salts disclosed herein increase the repressive state (NCOR1 recruitment) to a higher degree than previously disclosed PPARG modulators such as prior inverse agonists. Such results even arise in the mutant context. See e.g., the table qualitatively assessing NCOR1 recruitment and repression of PPARG target genes in HT1197 in the Exemplification section.

In some aspects, the compounds and pharmaceutical compositions described herein are useful in treating a disorder associated with PPARG function. Thus, provided herein are methods of treating a disorder associated with PPARG function, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof.

Also provided is the use of a compound described herein, or a pharmaceutically acceptable salt thereof, or a pharmaceutical composition comprising a disclosed compound or pharmaceutically acceptable salt thereof, for the manufacture of a medicament for treating a disorder associated with PPARG function. Also provided is a compound described herein, or a pharmaceutically acceptable salt thereof, or a pharmaceutical composition comprising a disclosed compound or pharmaceutically acceptable salt thereof, for use in treating a disorder associated with PPARG.

In one aspect, the disorder associated with PPARG is cancer. In some aspects, the cancer is associated with an up-regulated peroxisome proliferator-activated receptor (PPAR) signaling pathway. In some aspects, the up-regulated PPAR signaling pathway is associated with increased expression of one or more genes selected from Uroplakin 1A (UPK1A), Uroplakin 1B (UPK1B), Uroplakin (UPK2), Keratin 20 (KRT20), GATA Binding Protein 3 (GAT A3), Nuclear Receptor Corepressor 1 (NCOR1), Nuclear Receptor Corepressor 2 (NCOR2), Fatty Acid Binding Protein 4 (FABP4), Forkhead Box A1 (FOXA1), CD36 Molecule (CD36), Acyl-CoA Oxidase 1 (ACOX1), 3-Hydroxy-3-Methylglutaryl-CoA Synthase 2 (HMGCS2), Acyl-CoA Synthetase Long-Chain Family Member 5 (ACSL5), Arachidonate 5-Lipoxygenase (ALOX5), Acyl-CoA Synthetase Long-Chain Family Member 1 (ACSL1), and Angiopoietin Like 4 (ANGPTL4).

In some aspects, the cancer treated by the compounds, pharmaceutically acceptable salt thereof, and pharmaceutical compositions described herein is selected from breast cancer, pancreatic cancer, ovarian cancer, prostate cancer, renal cancer, bladder cancer, testicular cancer, urothelial cancer (e.g., non-muscle-invasive urothelial cancer, muscle-invasive urothelial cancer, metastatic urothelial cancer), skin cancer, melanoma, colon cancer, kidney cancer, brain cancer and a hematopoietic cancer (e.g., lymphoma, multiple myeloma and leukemia). In one aspect, the cancer treated by the compounds, pharmaceutically acceptable salt thereof, and pharmaceutical compositions described herein is urothelial cancer such as non-muscle-invasive urothelial cancer, muscle-invasive urothelial cancer, and metastatic urothelial cancer.

Other uses besides cancer are contemplated and include e.g., metabolic diseases (e.g., osteoporosis, rachitis, arthrosis, obesity, type I and type II diabetes mellitus), lipid metabolism disorder, pancreatitis, glucose metabolism disorder, diabetic neuropathy, diabetic complications, hyperuricemia, osteoporosis, rachitis, arthrosis inflammatory diseases (e.g., inflammatory skin diseases such as psoriasis, atopic dermatitis, eczema, acne vulgaris, other dermatitides and pruritus), pulmonary disorders (e.g., asthma and chronic obstructive pulmonary disease), autoimmune disease, neurodegenerative disease (e.g., multiple sclerosis, Alzheimer's disease, and Parkinson's disease), cardiovascular diseases (e.g., selected from atherosclerosis, venous and arterial occlusive diseases), restenosis after invasive procedures, cardiomyopathy, myocardial fibrosis, congestive heart failure, angiogenesis and neovascularization in neoplastic diseases and renal diseases.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

In some aspects, the pharmaceutical compositions are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### EXEMPLIFICATION

### Chemical Synthesis

The representative examples that follow are intended to help illustrate the present disclosure, and are not intended to, nor should they be construed to, limit the scope of the invention.

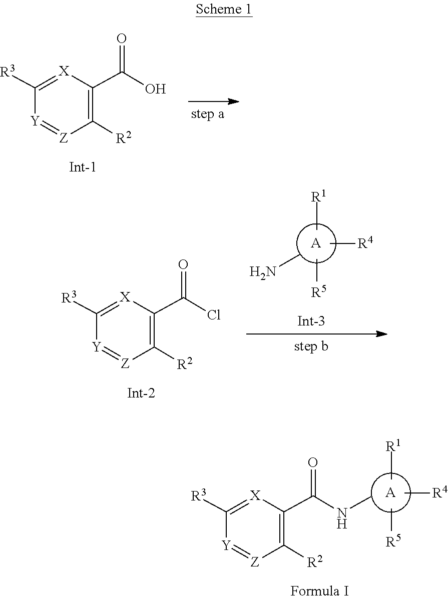

In step a, a compound having the formula Int-1 can be reacted with a chlorinating agent such as S(O)₂Cl to form the acid chloride (Int-2), which can then be reacted with a compound having the formula Int-3 to form the compound of Formula I, wherein X, Y, Z, R¹, R², R³, R⁴, and R⁵ for the above scheme are as defined herein.

The following prophetic compounds can be made according to the procedures described above.

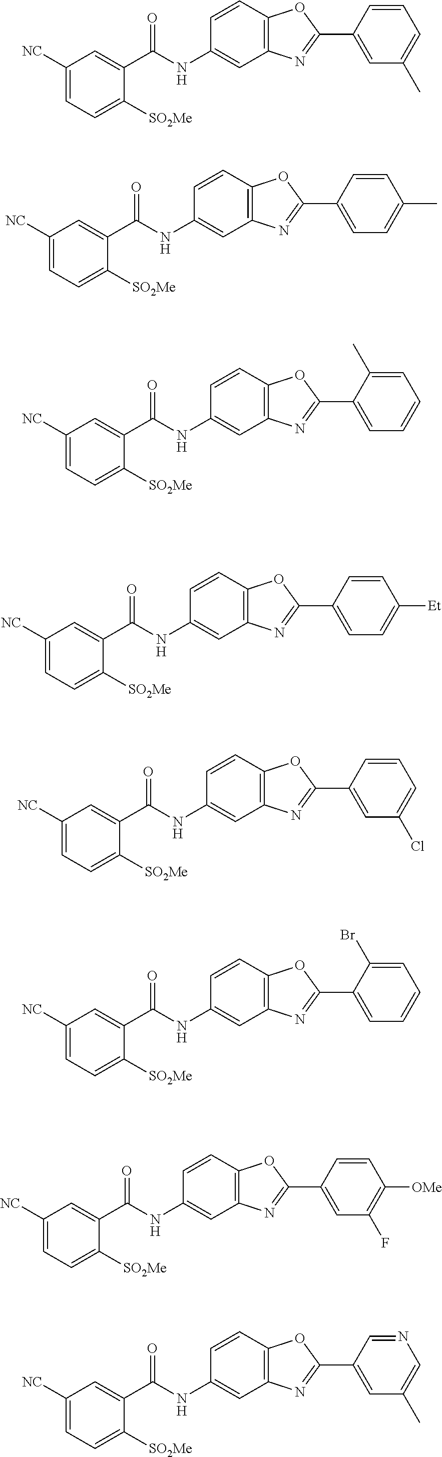

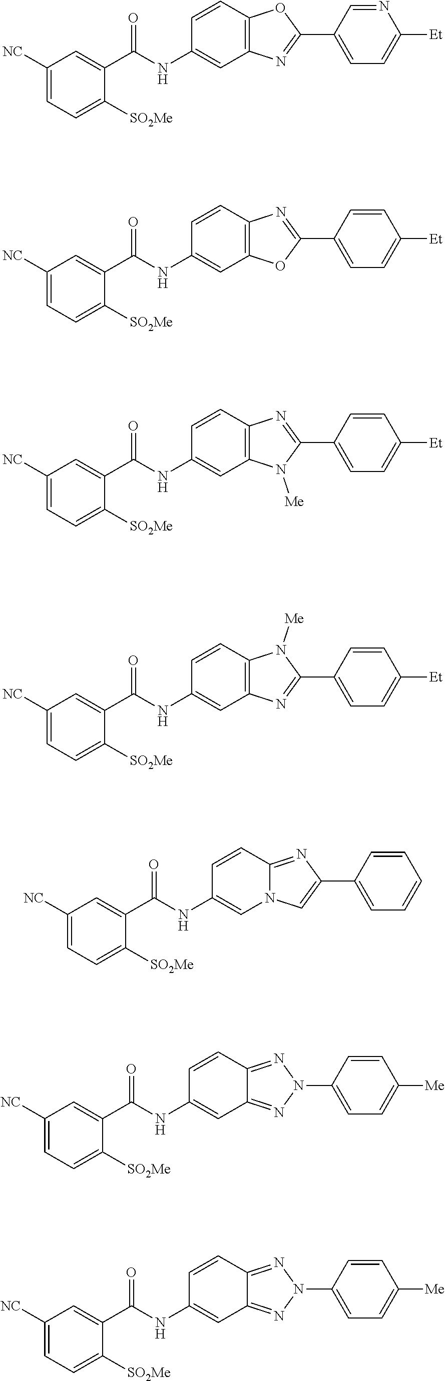

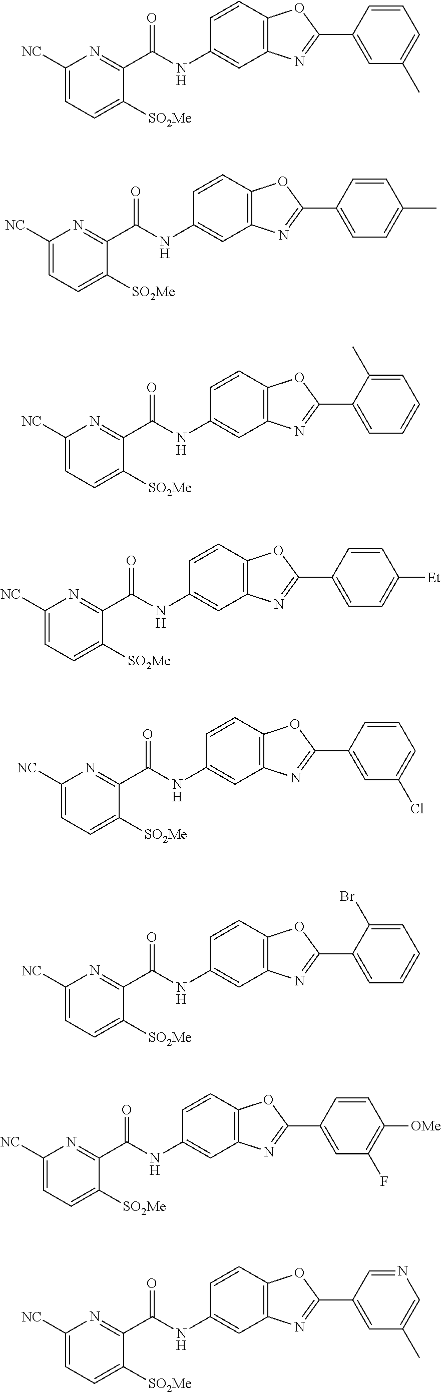

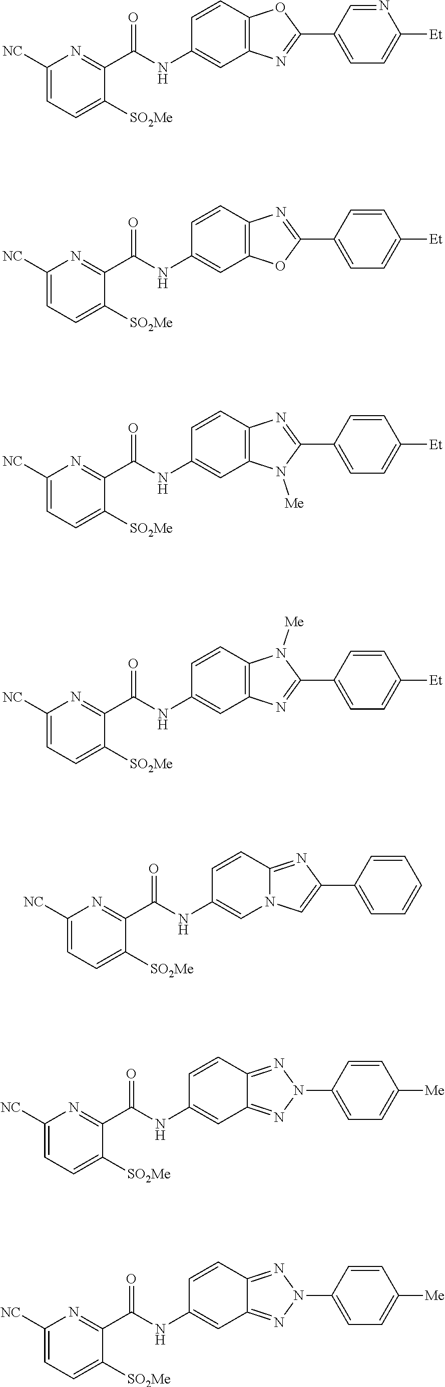

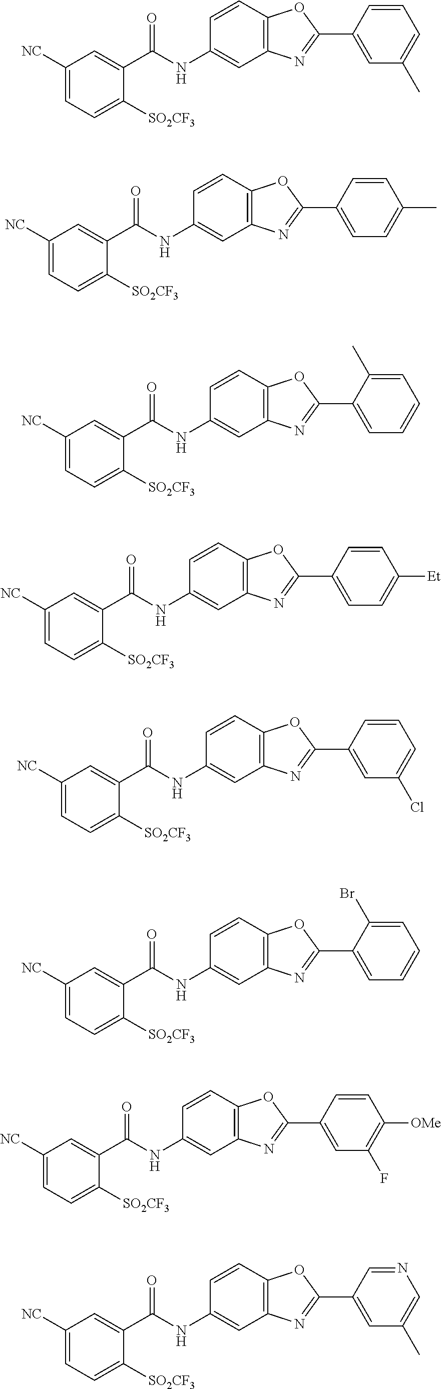

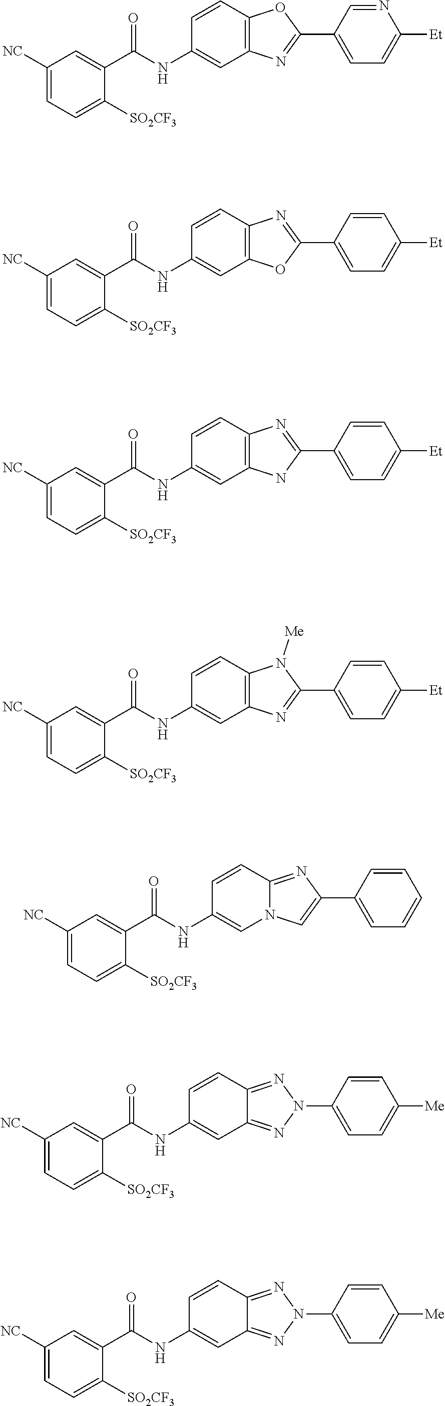

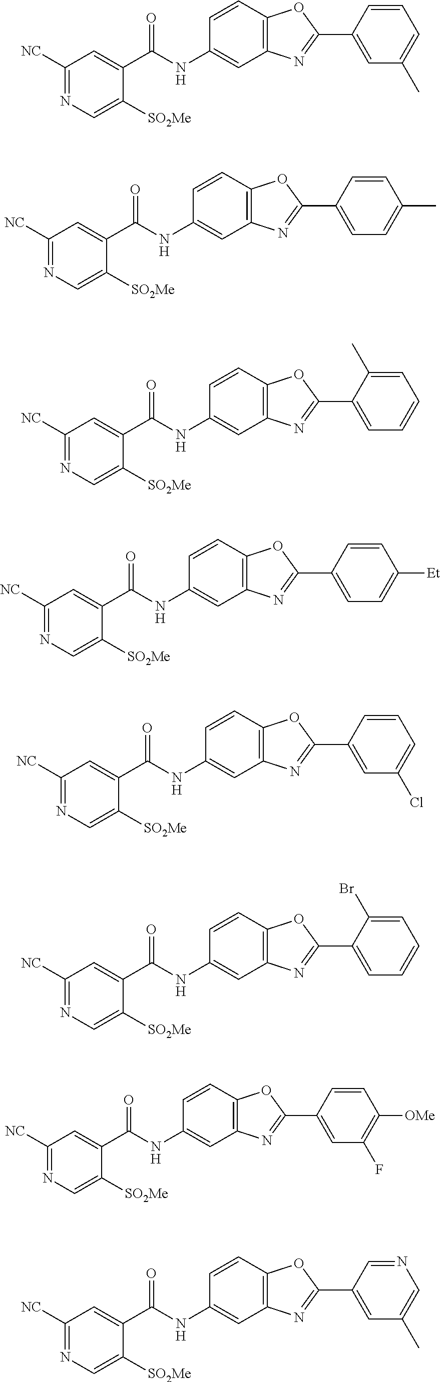

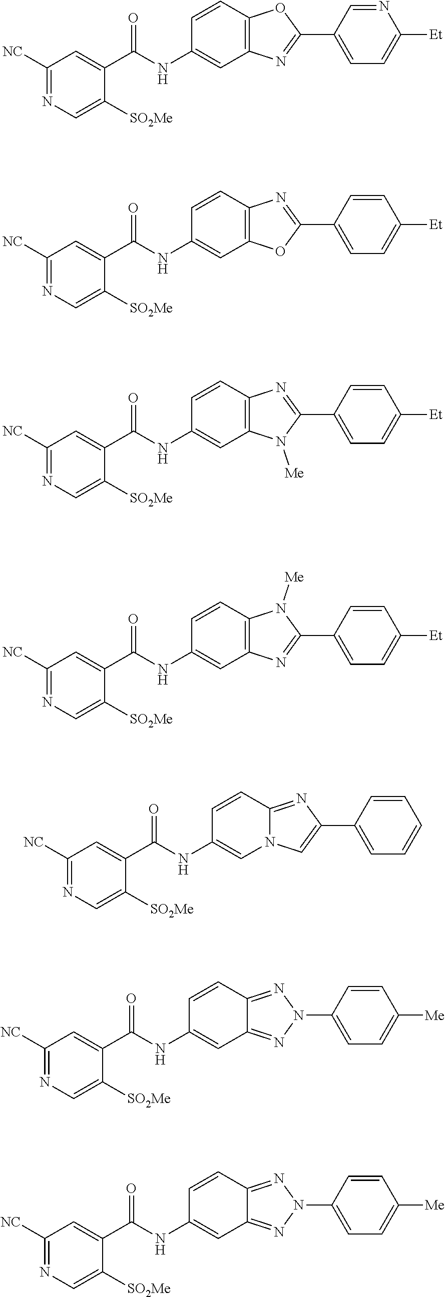

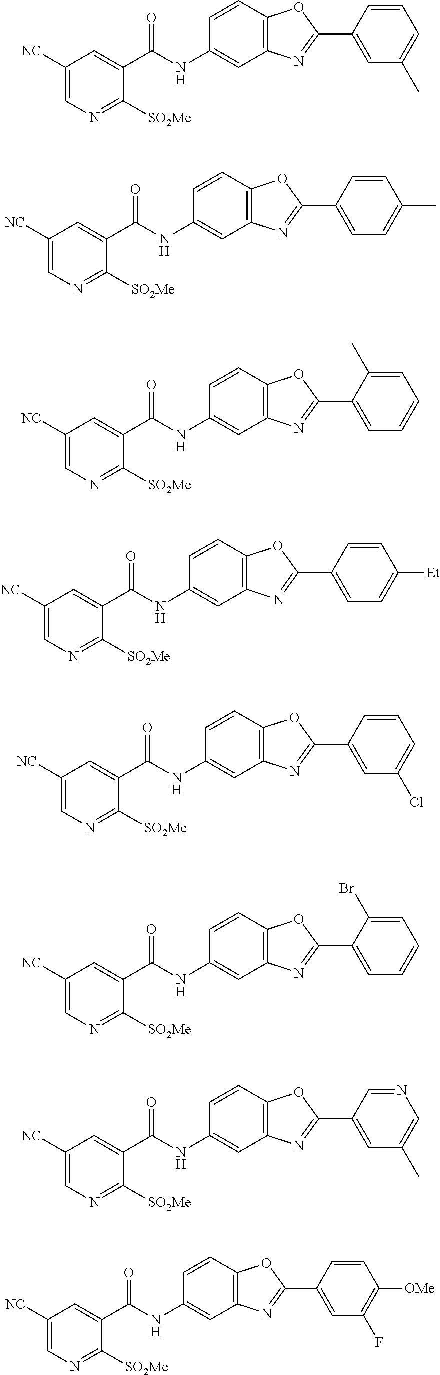

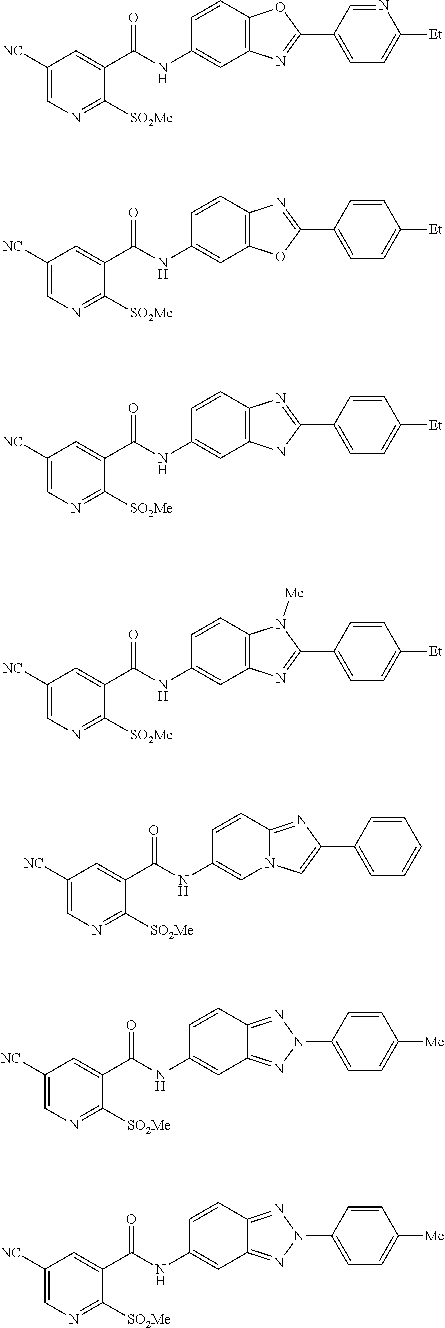

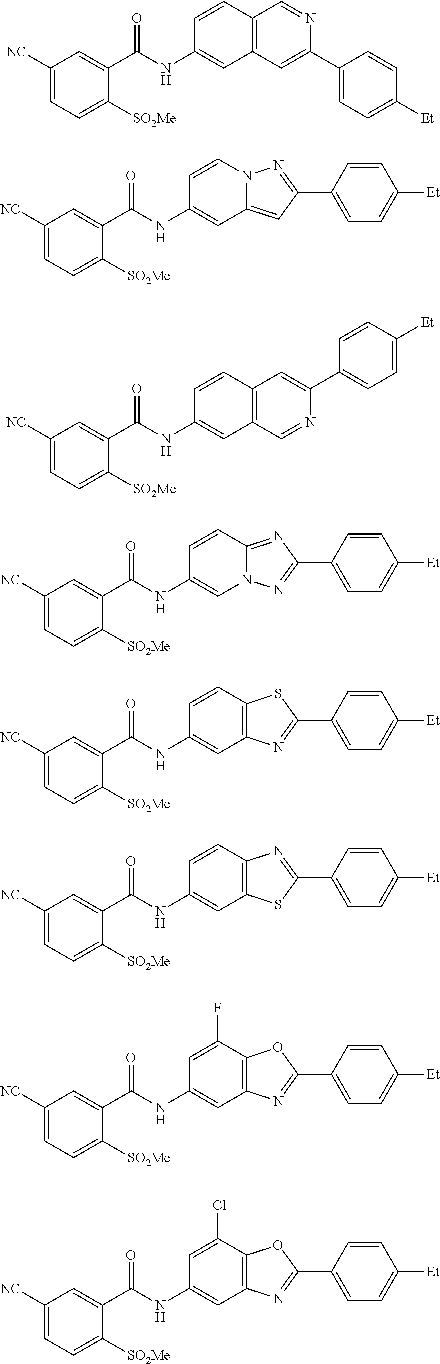

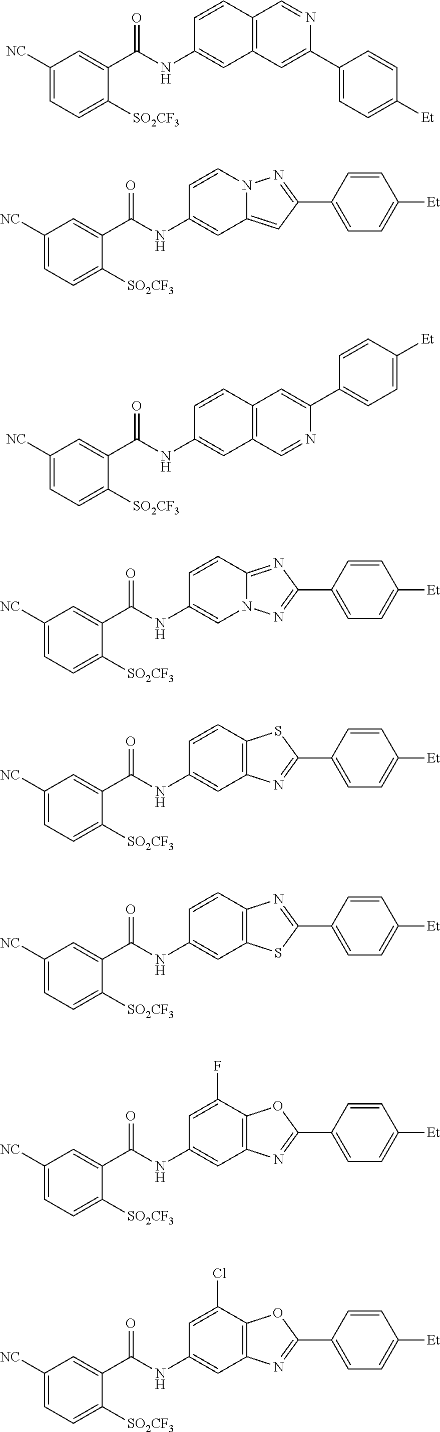

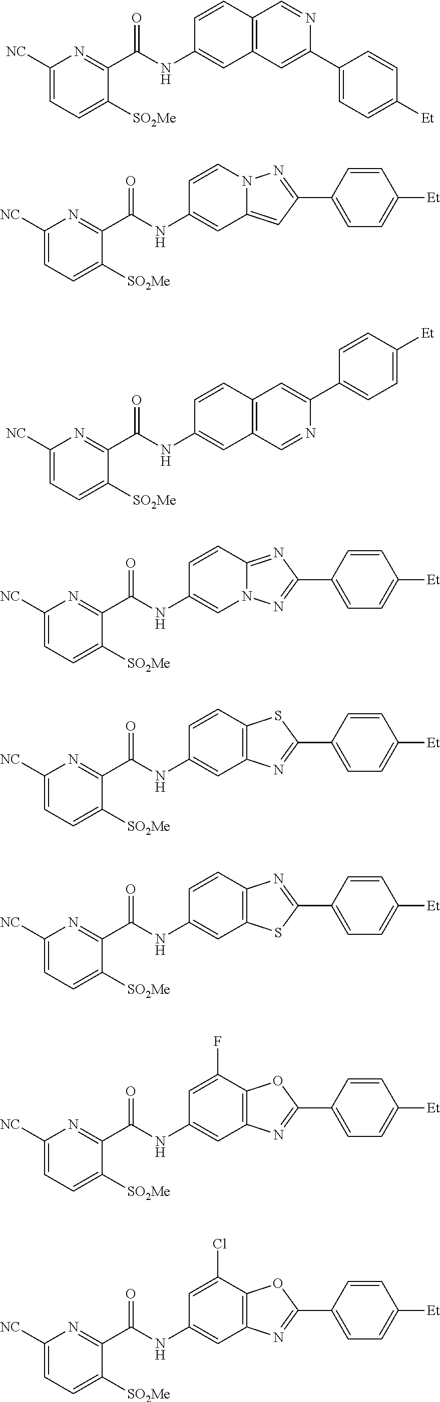

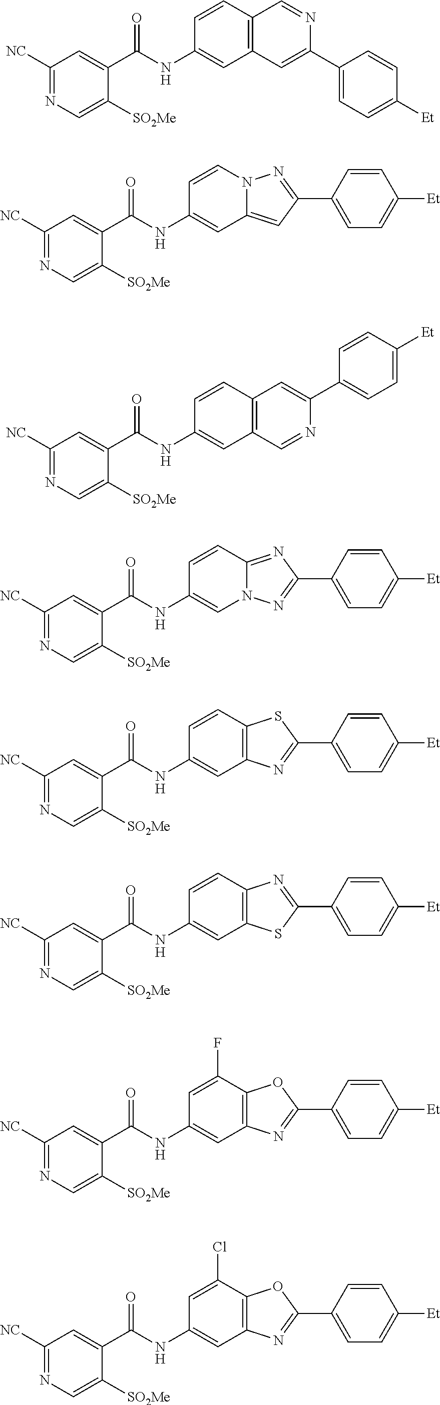

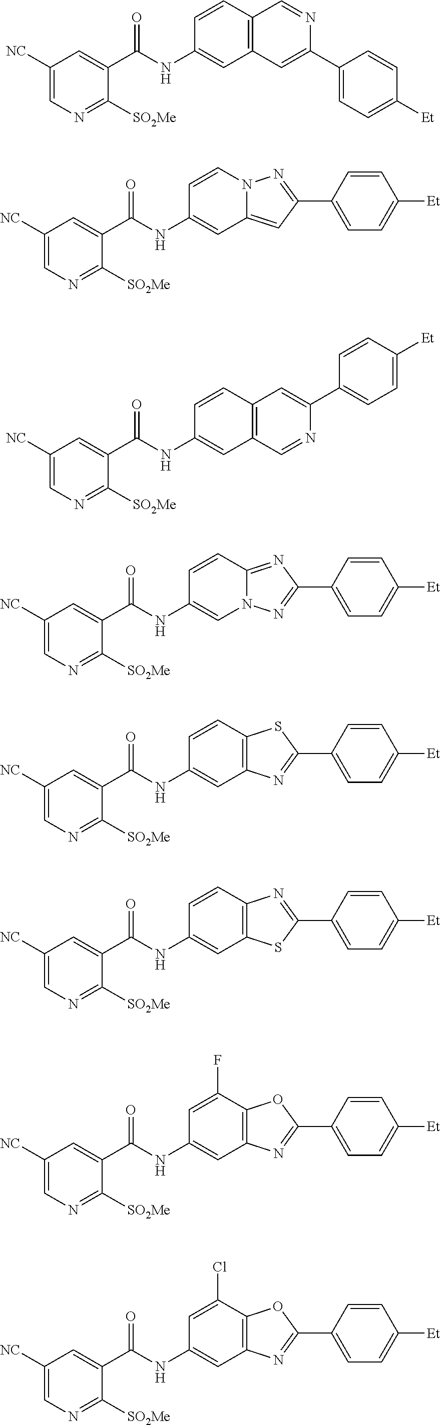

### Biochemical and Cellular Assays

### Compounds According to the Present Disclosure can be Tested as Follows:

### PPARγ-NCOR1 Recruitment Assay:

Compound potency (EC₅₀) and maximal extent of NCOR1 recruitment to PPARG can be assessed using a TR-FRET binding assay measuring association of a biotinylated NCOR1 ID2 peptide (Biotin-GHSFADPASNLGLEDIIRKALMG-amide)(SEQ ID NO: 1) to PPARG/RXRA LBD heterodimer.

### PPARγ-MED1 Blockade Assay:

Compound potency (IC₅₀) and maximal extent of MED1 repulsion to PPARG can be assessed a TR-FRET binding assay measuring association of a biotinylated MED1 LxxLL peptide (Biotin-VSSMAGNTKNHPMLMNLLKDNPAQ-amide)(SEQ ID NO: 2) to PPARG/RXRA LBD heterodimer.

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound having the Formula I: 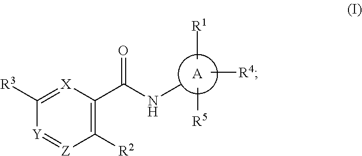
or a pharmaceutically acceptable salt thereof, wherein
X, Y, and Z are each independently N or —CR⁶;
ring A is a fused bicyclic heteroaryl;
R¹, R⁴, and R⁵ are each independently selected from hydrogen, halo, (C₁-C₄)alkyl, (C₁-C₄)alkoxy, halo(C₁-C₄)alkyl, halo(C₁-C₄)alkoxy, —(C₁-C₄)alkylOR^{a}, —(C₁-C₄)alkylC(O)R^{a}, —(C₁-C₄)alkylC(O)OR^{a}, —C(O)NR^{a}R^{b}, —(C₁-C₄)alkylC(O)NR^{a}R^{b}, —C(O)R^{a}, —C(O)OR^{a}, —NR^{a}R^{b}, —(C₁-C₄)alkylNR^{a}R^{b}, —C(O)NR^{a}SO₃H, —NR^{a}C(O)R^{b}, —NR^{a}C(O)OR^{b}, —NR^{a}C(S)OR^{b}, —N^{c}C(O)N^{a}R^{b}, —N^{c}C(S)NR^{a}R^{b}, —NR^{c}S(O)₂NR^{a}R^{b}, —C(S)R^{a}, —S(O)₂R^{a}, —S(O)R^{a}, —C(S)OR^{a}, —C(S)NR^{a}R^{b}, —NR^{a}C(S)R^{b}, —SR^{a}, phenyl, 4- to 6-membered heterocyclyl, and 5- to 7-membered heteroaryl, wherein each of said phenyl, 4- to 6-membered heterocyclyl, and 5- to 7-membered heteroaryl are optionally and independently substituted with 1 to 3 groups selected from R⁷;
R² is halo, —SR^{g}, —SOR^{g}, —SO₂R^{g}, —SO(═NR^{g})R^{h}, —OR^{g}, or —X¹Rⁱ;
R³ is cyano or nitro;
R⁶ is hydrogen, halo, (C₁-C₄)alkyl, (C₁-C₄)alkoxy, or hydroxyl;
R⁷ is selected from halo, (C₁-C₄)alkyl, halo(C₁-C₄)alkyl, (C₁-C₄)alkoxy, halo(C₁-C₄)alkoxy, nitro, oxo, cyano, —(C₁-C₄)alkylOR^{d}, —(C₁-C₄)alkylC(O)R^{d}, —(C₁-C₄)alkylC(O)OR^{d}, —C(O)NR^{d}R^{e}, —(C₁-C₄)alkylC(O)NR^{d}R^{e}, —C(O)R^{d}, —C(O)OR^{d}, —NR^{d}R^{e}, —(C₁-C₄)alkylNR^{d}R^{e}, —C(O)NR^{d}SO₃H, —N^{c}C(O)R^{e}, —N^{c}C(O)OR^{e}, —N^{c}C(S)OR^{e}, —N^{c}C(O)N^{d}R^{e}, —N^{c}C(S)N^{d}R^{e}, —NR^{f}S(O)₂NR^{d}R^{e}, —C(S)R^{d}, —S(O)₂R^{d}, —S(O)R^{d}, —C(S)OR^{d}, —C(S)NR^{d}R^{e}, —N^{c}C(S)R^{e}, and —SR^{d},
X¹ is S, SO, SO₂, or —SONH;
R^{a}, R^{b}, R^{c}, R^{d}, R^{e}, R^{f}, R^{g}, and R^{h} are each independently hydrogen, halo(C₁-C₄)alkyl, or a (C₁-C₄)alkyl optionally substituted with 1 or 2 —NR′R″ groups wherein R′ and R″ are each independently selected from hydrogen, (C₁-C₄)alkyl, and halo(C₁-C₄)alkyl; and
Rⁱ is (C₁-C₄)alkyl, (C₁-C₄)alkoxy, or halo(C₁-C₄)alkyl.

2. The compound of claim 1, wherein the compound is of the Formula II: 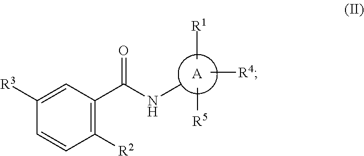
or a pharmaceutically acceptable salt thereof.

3. The compound of claim 1, wherein the compound is of the Formula III: 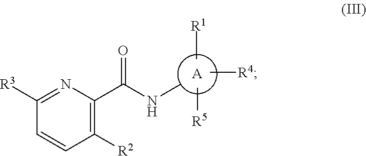
or a pharmaceutically acceptable salt thereof.

4. The compound of claim 1, wherein the compound is of the Formula IV: 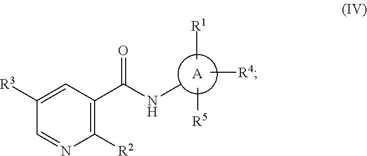
or a pharmaceutically acceptable salt thereof.

5. The compound of claim 1, wherein the compound is of the Formula V: 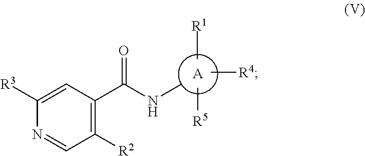
or a pharmaceutically acceptable salt thereof.

6. The compound of claim 1, or a pharmaceutically acceptable salt thereof, wherein R² is halo, —S(C₁-C₄)alkyl, —SO(C₁-C₄)alkyl, —SO₂(C₁-C₄)alkyl, or —O(C₁-C₄)alkylN[(C₁-C₄)alkyl]₂.

7. The compound of claim 1, or a pharmaceutically acceptable salt thereof, wherein R² is chloro, —SCH₃, —SOCH₃, —SO₂CH₃, or —O(CH₂)₂N(CH₃)₂.

8. The compound of claim 1, or a pharmaceutically acceptable salt thereof, wherein R² is chloro.

9. The compound of claim 1, or a pharmaceutically acceptable salt thereof, wherein R³ is cyano.

10. The compound of claim 1, or a pharmaceutically acceptable salt thereof, wherein ring A is selected from

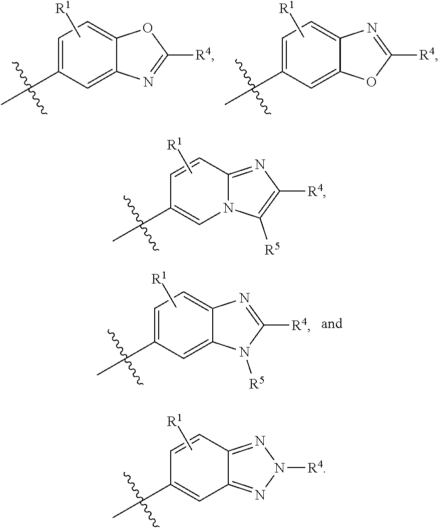

11. The compound of claim 1, or a pharmaceutically acceptable salt thereof, wherein ring A is

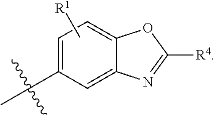

12. The compound of claim 1, or a pharmaceutically acceptable salt thereof, wherein:
(i) R¹ is hydrogen or halo;
(ii) R¹ is hydrogen, fluoro, or chloro; or
(iii) R¹ is hydrogen.

13-14. (canceled)

15. The compound of claim 1, or a pharmaceutically acceptable salt thereof, wherein:
(i) R⁵ is hydrogen or (C₁-C₄)alkyl; or
(ii) R⁵ is hydrogen or CH₃.

16. (canceled)

17. The compound of claim 1, or a pharmaceutically acceptable salt thereof, wherein R⁴ is phenyl or 5- to 7-membered heteroaryl, each optionally substituted with 1 to 3 groups selected from R⁷.

18. The compound of claim 1, or a pharmaceutically acceptable salt thereof, wherein R⁴ is phenyl or pyridinyl, each optionally substituted with 1 to 3 groups selected from R⁷.

19. The compound of claim 1, or a pharmaceutically acceptable salt thereof, wherein R⁴ is phenyl optionally substituted with 1 to 3 groups selected from R⁷.

20. The compound of claim 1, or a pharmaceutically acceptable salt thereof, wherein R⁷ is selected from halo, (C₁-C₄)alkyl, and (C₁-C₄)alkoxy.

21. The compound of claim 1, or a pharmaceutically acceptable salt thereof, wherein R⁷ is selected from bromo, fluoro, CH₃, CH₂CH₃, and OCH₃.

22. A pharmaceutical composition comprising a compound of claim 1, or a pharmaceutically acceptable salt thereof; and a pharmaceutically acceptable carrier.

23. A method of treating a cancer responsive to the suppression of PPARG in a subject, comprising administering to the subject a therapeutically effective amount of a compound of claim 1, or a pharmaceutically acceptable salt thereof.


SEQUENCE LISTING (2 sequences)

SEQ ID NO: 1 - protein, 23 aa, synthetic construct
  REGION 1..23: note = Synthetic: biotinylated NCOR1 ID2 peptide
  MOD_RES 1: note = Biotin
  MOD_RES 23: note = Amide
GHSFADPASNLGLEDIIRKALMG

SEQ ID NO: 2 - protein, 24 aa, synthetic construct
  REGION 1..24: note = Synthetic: biotinylated MED1 LxxLL peptide
  MOD_RES 1: note = Biotin
  MOD_RES 24: note = Amide
VSSMAGNTKNHPMLMNLLKDNPAQ